(19) United States
(12) Patent Application Publication
(10) Pub. No.: US 2026/0102479 A1
(43) Pub. Date: Apr. 16, 2026

(54) METHOD FOR THE PURIFICATION OF CAPSULAR POLYSACCHARIDES

(71) Applicant: BIOLOGICAL E LIMITED, Hyderabad (IN)

(72) Inventors: Chandra Shekar MACHA, Hyderabad (IN); Eswara Reddy KOWLAKUNTLA, Hyderabad (IN); Sudeep KUMAR, Hyderabad (IN); Vikram Madhusudan PARADKAR, Hyderabad (IN); Narender Dev MANTENA, Hyderabad (IN); Mahima DATLA, Hyderabad (IN)

(21) Appl. No.: 19/114,705

(22) PCT Filed: Sep. 19, 2023
(86) PCT No.: PCT/IN2023/050869

§ 371 Date: Mar. 24, 2025

(30) Foreign Application Priority Data
Sep. 19, 2022 (IN) 202241053370

(51) Int. Cl. A61K 39/09; A61K 39/00; A61K 39/095; A61K 39/102; A61K 39/108; A61K 39/385; B01D 71/10; C08B 37/00; C12N 1/06; C12N 1/20; C12R 1/46

(52) U.S. Cl. CPC A61K 39/092; A61K 39/0266; A61K 39/095; A61K 39/102; A61K 39/385; B01D 71/10; C08B 37/0003; C12N 1/06; C12N 1/20; A61K 2039/6037; C12R 2001/46

(57) ABSTRACT

The present invention broadly relates to the improved method for purification of capsular polysaccharides. Particularly, the present invention relates to the method of purification of capsular polysaccharides from Streptococcus pneumoniae and other similar related capsular polysaccharides produced from both gram-negative and gram-positive microorganisms. More particularly, the invention involves passing the crude capsular polysaccharide solution through reinforced cellulose membrane having quaternary ammonium, and further contacting the solution with silicon dioxide (SiO2) and isolating the capsular polysaccharide in a pure form. The purified capsular polysaccharide is useful for producing vaccines that contain the polysaccharide alone or are conjugated to proteins.

### FIELD OF INVENTION

The present invention broadly relates to the improved method for purification of capsular polysaccharides. Particularly, the present invention relates to the method of purification of capsular polysaccharides from Streptococcus pneumoniae and other similar related capsular polysaccharides produced from both gram-negative and gram-positive microorganisms. More particularly, the invention involves passing the crude capsular polysaccharide solution through reinforced cellulose membrane having quaternary ammonium, and further contacting the solution with silicon dioxide (SiO₂) and isolating the capsular polysaccharide in a pure form. The purified capsular polysaccharide is useful for producing vaccines that contain the polysaccharide alone or are conjugated to proteins.

### BACKGROUND

The process of manufacture of vaccine is particularly critical at every stage to determine it is safe for human use. Polysaccharides are carbohydrates used in several industrial applications, such as thickeners, gellants, emulsifiers, and delivery systems of many commercial products. The capsular polysaccharides present on microbial cells may be used as a component of immunization. Upon immunization with purified capsular polysaccharides in a formulated composition it prevents against disease causing organisms like Neisseria meningitidis, Streptococcus pneumoniae, Haemophilus influenzae type b, and Salmonella typhi by inducing the respective immune response. This has led to them being an important component in the design of vaccines.

Capsular polysaccharides have also proven useful in eliciting immune responses especially when conjugated to carrier proteins. Conjugated vaccines trigger improved immunogenic responses including in children and immune compromised individuals and, in elderly population. The polysaccharide conjugated with proteins like CRM197, tetanus toxoid, diphtheria toxoid, and other surface proteins are well proven and highly immunogenic.

All such immunogenic or vaccine preparations approved for human use require capsular polysaccharides in highly purified forms. Capsular polysaccharides are present on the outer surface of bacterial cells. During separation of capsular polysaccharides from the cell there is release of cellular components like nucleic acid, proteins, cell wall etc. In addition to biosynthetic products produced during fermentation, media nutrients also contribute contaminants. The process for the isolation/purification of capsular polysaccharide involves multiple steps ranging from chromatography, filtration, treatment with detergents, solvents, enzymes to hydrolyze the nucleic acid, protein, polysaccharide etc.

The following references disclose various methods for the removal of protein and other impurities from capsular polysaccharides.

Canadian patent No. CA1206905 describes a method which uses toxic and organic solvents like Phenol, butanol, Toluene and chloroform and use of detergent Cetavion (CTAB).

European Patent No. EP0497525 discloses the method which involves thermal hydrolysis and sodium acetate to hydrolyze the sample.

U.S. Pat. No. 5,847,112 mentions a method which makes use of multiple isopropyl alcohol precipitations and Cetavlon for the precipitation of polysaccharide.

US Patent Publication No. 20060228380 discloses a method which comprises Cetavlon for polysaccharide precipitation, carbon filter for nucleic acid removal and potassium iodide for precipitation of Cetavlon.

WO Publication No. 2006/082527 A2 discloses a purification process for the capsular polysaccharide of S. agalactiae in which the saccharide is initially treated with an aqueous mixture of an alcohol and a calcium salt, followed by precipitation with a cationic detergent.

IN 1572/MUM/2010 discloses a purification process for removal of protein contaminants which involves treatment of nuclease treated polysaccharide solution with a mixture of detergent & saline; followed by centrifugation, diafiltration, and chromatography.

WO Publication No. 2012/127485 A1 discloses an alcohol and CTAB free method for the purification of pneumococcal polysaccharides which utilizes chromatographic separation of capsular polysaccharides (PnPs) on the basis of differences in their net surface charge.

Referring to the prior arts, it is observed that it is very challenging to remove process impurities from capsular polysaccharides as both are negatively charged.

The processes disclosed in the above discussed prior art involve multiple and complex steps, which adversely affect yield, quality, stability, processing time and process operations. Further, these processes are expensive to run, require a high degree of skill to perform, and a significant amount of time to reduce impurity levels.

Also, the processes disclosed in the prior art are not efficient in removing the process impurities from capsular polysaccharides of bacteria if the initial load is high and if produced in aggregate forms. Hence, there is a need for improved methods for the removal of impurities from complex cellular lysates. During their continuous efforts, inventors have found that the use of reinforced cellulose membrane with quarternary ammonium ligand along with Silicon dioxide effectively removes impurities. Optimized pH ranges are used in order to bind highly negatively charged DNA, endotoxins, host cell proteins with reinforced cellulose membrane having quaternary ammonium as ligand.

The method developed by the inventors is a simple, efficient and one that could be easily scaled up.

### Objective of the Invention

An important objective of the present invention is to provide an improved method to purify capsular polysaccharides from Streptococcus pneumoniae and other similar related capsular polysaccharides produced from both gram-negative and gram-positive microorganisms and provide purified capsular polysaccharides meeting the compendia requirements.

Yet another objective of the present invention is to provide a simple and cost-effective method to purify capsular polysaccharides, without the use of complex and expensive chromatography methods and toxic reagents like phenol.

Furthermore, another important objective of the present invention to provide a process to purify capsular polysaccharides robustly at a commercial scale, requiring low-cost materials in the process and no specialized facilities or disposal of hazardous materials.

### SUMMARY OF THE INVENTION

The present invention provides a method for purification of capsular polysaccharides, wherein the said method comprises the steps of:
a. culturing the bacterial cells in standard growth conditions to obtain a fermentation broth;
b. lysing the bacterial cells in fermentation broth obtained in step (a) to obtain a bacterial cell lysate comprising capsular polysaccharide, protein, nucleic acids, cell wall components and other impurities;
c. subjecting the bacterial cell lysate from step (b) to sizing using homogenizer to obtain a sized crude capsular polysaccharide solution;
d. passing the sized crude capsular polysaccharide obtained in step (c), through a reinforced cellulose membrane having quaternary ammonium as ligand; and
e. contacting the solution obtained after passing through the reinforced cellulose membrane having quaternary ammonium, in step (d), with SiO₂ and isolating the capsular polysaccharide in a pure form.

In an embodiment, the method of the present invention does not require the use of complex and expensive chromatography methods and toxic reagents like phenol to purify capsular polysaccharides.

In another embodiment of the present invention, the capsular polysaccharide is isolated from Streptococcus pneumoniae serotypes selected from serotypes 1 and 5.

In yet another embodiment, the purification method of the present invention is an alcohol-free process and does not use CTAB or other harsh chemicals.

### DETAILED DESCRIPTION OF THE INVENTION

Embodiments described herein can be understood more readily by reference to the following detailed description, and examples. Elements and methods described herein are merely illustrative of the principles of the present invention and are not limited to the specific embodiments presented in the detailed description, and examples. Numerous modifications and adaptations will be readily apparent to those of skill in the art without departing from the spirit and scope of the invention.

Unless defined otherwise, all technical and scientific terms used herein have the same meaning as commonly understood by one of ordinary skill in the art to which the methods belong. Although any method, device, kit, reagent or composition similar to or equivalent to those described herein can also be used in the practice or testing of the methods, representative illustrative methods and compositions are now described. It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements or use of a “negative” limitation. The term “sized” or “sizing” as used herein refers to reducing the size of a native polysaccharide by various methods.

A “lytic agent” is any agent that aids in cell wall breakdown including, for example, detergents. As used herein, the term “detergent” refers to any anionic or cationic detergent capable of inducing lysis of bacterial cells.

The terms “exposing”, or “contacting” means incubation of capsular polysaccharide preparation with other components to treat the sample for the removal of impurities, to make pure polysaccharide.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The practice of the present invention will employ, unless indicated specifically to the contrary, conventional methods of virology, immunology, microbiology, and molecular biology, within the skill of the art. Such techniques are explained fully in the available literature.

To remove process impurities from capsular polysaccharides is a challenging step as both the impurities and the capsular polysaccharides are negatively charged. The pH value at which a biomolecule has no net charge is the isoelectric point. Accordingly, in the present invention optimized pH ranges are used in order to bind highly negatively charged DNA, endotoxins, host cell proteins with reinforced cellulose membrane having quaternary ammonium as ligand.

The present invention provides an improved method for purification of capsular polysaccharides, wherein the source of capsular polysaccharide is gram-negative and gram-positive microorganisms. The said improved method involves passing the crude capsular polysaccharide solution through reinforced cellulose membrane having quaternary ammonium, and further contacting the solution with SiO₂. The resulting solution after passing through reinforced cellulose membrane having quaternary ammonium and further exposing to SiO₂, is enriched in polysaccharide and reduced in one or more impurities such as protein, nucleic acid, cell wall polysaccharide, and other cell derived materials.

The capsular polysaccharides obtained according to the present invention are in (substantially) pure form.

An important aspect of the present invention relates to the removal of impurities from complex bacterial cellular lysates comprising crude capsular polysaccharide. It involves the use of reinforced cellulose membrane with quaternary ammonium ligand along with silicon dioxide resulting in effective removal of impurities. Importantly, remarkable reduction in the process impurities without losing the capsular polysaccharide was observed.

In another aspect of the present invention, purified capsular polysaccharide met all the release test specifications as per world health organization—technical report series—927. Further, the WHO Specifications for purified capsular polysaccharides are as follows:
Protein Content: Generally, not more than 2% of dry weight polysaccharide, depending on the serotype polysaccharide. The level is not more than 2% for Serotype 1 and not more than 3% for Serotype 5.
Nucleic acid Content: Not more than 2% of dry weight polysaccharide.
Endotoxin content: Less than 0.5 IU/μg of polysaccharide.

Some of the important embodiments of the present invention are as below:

An important embodiment of the present invention relates to a method for purification of crude capsular polysaccharides, wherein the said method comprises steps of:
i. passing a solution of bacterial cell lysate comprising capsular polysaccharide, protein, nucleic acids, cell wall components and other impurities through a reinforced cellulose membrane having quaternary ammonium as ligand; and
ii. contacting the solution obtained after passing through the reinforced cellulose membrane having quaternary ammonium, in step (i), with SiO₂ and isolating the capsular polysaccharide in a pure form.

A preferred embodiment of the present invention relates to a method for purification of crude capsular polysaccharides, wherein the said method comprises steps of:
a. culturing the bacterial cells in standard growth conditions to obtain a fermentation broth;
b. lysing the bacterial cells in fermentation broth obtained in step (a) to obtain a bacterial cell lysate comprising capsular polysaccharide, protein, nucleic acids, cell wall components and other impurities;
c. subjecting the bacterial cell lysate from step (b) to sizing using homogenizer to obtain a sized crude capsular polysaccharide solution;
d. passing the sized crude capsular polysaccharide solution obtained in step (c), through a reinforced cellulose membrane having quarternary ammonium as ligand; and
e. contacting the solution obtained after passing through the reinforced cellulose membrane having quaternary ammonium, in step (d), with SiO₂ and isolating the capsular polysaccharide in a pure form.

In an embodiment, the capsular polysaccharide of the present invention is isolated from bacteria.

In an embodiment of the invention, the bacteria is a gram-positive bacteria. In one aspect, the bacteria is selected from but not limited to Streptococcus, Staphylococcus, Enterococci, Bacillus, Corynebacterium, Listeria, Erysipelothrix, or Clostridium. In a further aspect, the bacteria is selected from but not limited to Streptococcus pneumoniae, Streptococcus pyogenes, Streptococcus agalactiae, Group C & G Streptococcii or Staphylococcus aureus.

In another embodiment of the invention, the bacteria is a gram-negative bacteria. In one aspect, the bacteria is selected from but not limited to Haemophilus, Neisseria, or Klebsiella. In another aspect, the bacteria is selected from but not limited to Haemophilus influenzae, Neisseria meningitidis, or Klebsiella pneumoniae.

In another important embodiment of the present invention, wherein the bacteria is selected from but not limited to Streptococcus, Staphylococcus, Enterococci, Bacillus, Corynebacterium, Listeria, Erysipelothrix, Clostridium, Haemophilus, Neisseria, or Klebsiella.

In yet another embodiment of the present invention, wherein the bacteria is selected from but not limited to Streptococcus pneumoniae, Streptococcus pyogenes, Streptococcus agalactiae, Group C & G Streptococcii Staphylococcus aureus, Haemophilus influenzae, Neisseria meningitidis, or Klebsiella pneumoniae.

In another embodiment of the present invention, wherein the capsular polysaccharide is isolated from, but not limited to Streptococcus pneumoniae serotypes selected from the group comprising of serotypes 1, 2, 3, 4, 5, 6A, 6B, 6C, 6D, 6E, 6G, 6H, 7A, 7B, 7C, 7F, 8, 9A, 9L, 9F, 9N, 9V, 10A, 10B, 10C, 10D, 10F, 11A, 11F, 11B, 11C, 11D, 11E, 12A, 12B, 12F, 13, 14, 15A, 15C, 15B, 15F, 16A, 16F, 17A, 17F, 18C, 18F, 18A, 18B, 19A, 19B, 19C, 19F, 20, 20A, 20B, 21, 22A, 22F, 23A, 23B, 23F, 24A, 24B, 24F, 25F, 25A, 27, 28F, 28A, 29, 31, 32A, 32F, 33A, 33C, 33D, 33E, 33F, 33B, 34, 45, 38, 35A, 35B, 35C, 35F, 36, 37, 38, 39, 40, 41F, 41A, 42, 43, 44, 45, 46, 47F, 47A, and 48.

In an exemplary embodiment of the present invention, the capsular polysaccharide is isolated from Streptococcus pneumoniae serotypes selected from serotypes 1 and 5.

In another embodiment of the present invention, the improved purification method is an alcohol-free process and does not use CTAB or other harsh chemicals.

In an embodiment of the present invention, capsular polysaccharides are produced by growing the bacteria in a medium (e.g., a solid or preferably a liquid medium).

In another embodiment of the present invention, the starting material for methods of the present invention is a bacterial culture and preferably a liquid bacterial culture (e.g., a fermentation broth).

In an exemplary embodiment bacterial culture is grown in liquid MH medium. Bacterial culture is grown in fermenter under controlled parameters like temperature in the range of 35±2° C., pH in the range of 7.1±0.3 and back pressure NMT in the range of 0.2-1.0 Bar, Optical density is monitored at every 30-60 minutes intervals. Further, fermentation is stopped when culture enters into stationary or decline phase. Thereafter, the capsular polysaccharides are then prepared by treating the bacterial cells using various processes such as, but not limited to, cell lysis, centrifugation, depth filtration, concentration/diafiltration operations, and precipitation steps etc.

In an embodiment, the fermentation broth is inactivated and lysed to obtain bacterial cell lysate. Cell debris is removed from the inactivated fermentation broth using continuous centrifuge. Supernatant is depth filtered and subjected to concentration and diafiltration using phosphate buffer and optionally subjected to ultrafiltration. The solution obtained after the preliminary treatment of the bacterial cell lysate is referred to as crude polysaccharide solution.

In an embodiment, the fermented broth is lyzed using a lytic agent.

In a preferred embodiment of the present invention, the lytic agent is selected from, but not limited to sodium deoxycholate (DOC), N-lauryl sarcosine (NLS), chenodeoxycholic acid sodium, and saponins.

In another embodiment of the present invention, the fermented broth is treated with 0.1-0.5% w/v DOC solution to lyse the cells in the fermentation broth.

In an embodiment, the capsular polysaccharides in the crude capsular polysaccharide solution or the bacterial cell lysate are sized before passing the solution through a reinforced cellulose membrane having quaternary ammonium as ligand. The solution obtained after subjecting the crude polysaccharide solution or the bacterial cell lysate to sizing is referred to as sized crude polysaccharide solution.

In another embodiment, to avoid the cross linking of process impurities with product impurities and make the capsular polysaccharide tough for sizing, high molecular weight product aggregates in the bacterial cell lysate comprising capsular polysaccharide are removed by introducing the sizing step using the chemical sizing methods or treatment with high-pressure homogenizer, prior to the purification process.

In an exemplary embodiment, the capsular polysaccharides are size reduced by various mechanical means known in the art such as high-pressure techniques such as microfluidization, Emulsiflex™, high-pressure homogenization, sonication or Gaulin homogenization. Homogenization achieves high shear rates by pumping the process stream through a flow path with sufficiently small dimensions. The shear rate is increased by using a larger applied homogenization pressure, and exposure time is increased by recirculating the feed stream through the homogenizer.

In an embodiment, the homogenization is carried out at a pressure of at least 500 bar, until the polysaccharide of desired size is obtained.

In an embodiment of the present invention, homogenization is preferably performed at 500-1000 Bar at flow rate of 50-200 L/hr.

In an embodiment, the molecular weight of the sized polysaccharide ranges from 50 kDa to 1000 kDa.

In another embodiment, the molecular weight of the sized polysaccharide ranges from about 400 kDa to 700 kDa.

In a preferred embodiment, the molecular weight of the sized polysaccharide ranges from about 150 kDa to 350 kDa.

In an embodiment, the sized crude capsular polysaccharide solution is passed through reinforced cellulose membrane having quaternary ammonium ligand.

In an embodiment, the reinforced cellulose membrane having quaternary ammonium as ligand is be used in any of the formats like matrix or filter capsule.

In an embodiment, the reinforced cellulose membrane having quaternary ammonium ligand is initially cleaned with an alkali and subsequently equilibrated with a buffer prior to use.

In an embodiment, the membrane is initially sanitized with IN NaOH for not less than 30 minutes.

In another embodiment of the present invention, reinforced cellulose membrane having quaternary ammonium ligand sanitization is done using 0.5M NaOH and subsequently equilibration is done using phosphate buffered saline solution.

In another embodiment of the present invention, the reinforced cellulose membrane having quaternary ammonium ligand is equilibrated with buffer till pH reaches 6.5-7.5 and conductivity reaches 2-4 mS/cm.

In yet another embodiment, the reinforced cellulose membrane having quaternary ammonium ligand is equilibrated with buffer till pH reaches 6.5-6.8 and conductivity reaches to 3.2±0.3 mS/cm.

In an embodiment, the pH of the crude sized capsular polysaccharide solution is adjusted to a range of about 6.0 to 10.0 before passing the solution through reinforced cellulose membrane having quaternary ammonium ligand.

In a preferred embodiment, the pH of the sized crude capsular polysaccharide solution is adjusted to a range of about 6.5 to 6.8 before passing the solution through reinforced cellulose membrane having quaternary ammonium ligand.

In an embodiment of the present invention, the conductivity of the sized crude capsular polysaccharide solution is adjusted to a range of about 1.0 mS/cm to 5.0 mS/cm before passing the solution through reinforced cellulose membrane having quaternary ammonium ligand.

In a preferred embodiment of the present invention, the conductivity of the sized crude capsular polysaccharide solution is adjusted to a range of about 2.8 to 3.2 mS/cm before passing the solution through reinforced cellulose membrane having quaternary ammonium ligand.

In an embodiment of the present invention, the conductivity of the sized crude capsular polysaccharide solution is adjusted using 0.5-5M NaCl or 0.5-5M KCl solution.

In an embodiment of the present invention, the sized crude capsular polysaccharide solution is passed through the membrane at a flow rate ranging from about 2 L/min to 10 L/min.

In a preferred embodiment of the present invention, the sized crude capsular polysaccharide solution is passed through the membrane at a flow rate ranging from about 3 L/min to 5 L/min.

In another embodiment of the present invention, the sized crude capsular polysaccharide solution is passed through the reinforced cellulose membrane at a flow rate of 2-5 L/min.

In an embodiment of the present invention, the flow-through solution, obtained upon passing the sized crude capsular polysaccharide solution through reinforced cellulose membrane having quaternary ammonium ligand, is exposed, or contacted with SiO₂.

In an embodiment, the residual SiO₂, after contacting, is removed by centrifugation.

In an embodiment of the present invention, the solution obtained upon passing the sized crude capsular polysaccharide solution through reinforced cellulose membrane having quaternary ammonium ligand is subsequently exposed or contacted with SiO₂, without any intermediary treatment or purification process.

In an embodiment, the SiO₂ used is in different forms/particle size such as fine particles ranging from 0.01 μm to 200 μm.

In another embodiment, the SiO₂ used is in different forms/particle size such as fine particles preferably in the range of 3 μm to 40 μm.

In a preferred embodiment, the SiO₂ particle size is in the range of 10 μm to 80 μm, and more preferably in the range of 20 μm to 60 μm.

In an embodiment of the present invention, the amount of SiO₂ used may range from 0.5 to 20% (w/v).

In a preferred embodiment, the amount of SiO₂ ranges from 5% to 7% (w/v) (50 g/L to 70 g/L)

In an embodiment, SiO₂ used is optionally prepared by heating above 60° C. and for at least 1 hour and cooling prior to use.

In another embodiment, the SiO₂ used may be pyrogenated or depyrogenated.

In another embodiment, the pH of the solution obtained upon passing the sized crude capsular polysaccharide solution through reinforced cellulose membrane having quaternary ammonium ligand is maintained in the range from acidic region to alkaline region, and preferably from 3.0 to 9.0, while contacting SiO₂.

In another embodiment, the pH of the solution obtained upon passing the sized crude capsular polysaccharide solution through reinforced cellulose membrane having quaternary ammonium ligand is adjusted using acids such as acetic acid, phosphoric, formic acid, hydrochloric acid and the like and alkalis such as sodium, potassium or ammonium hydroxide and the like.

In a preferred embodiment, the pH of the solution obtained upon passing the sized crude capsular polysaccharide solution through reinforced cellulose membrane having quaternary ammonium ligand is maintained in the range from 5.0 to 7.0, while contacting with SiO₂.

In an embodiment, contact or exposure of the solution obtained upon passing the sized crude capsular polysaccharide solution through reinforced cellulose membrane having quaternary ammonium ligand, to SiO₂ is carried out at a temperature ranging from 15° C. to 60° C. for a period of 10 minutes to 16 hours.

In another embodiment, contact or exposure of the solution obtained upon passing the sized crude capsular polysaccharide solution through reinforced cellulose membrane having quaternary ammonium ligand, to SiO₂ is carried out at room temperature (22±4° C.) for a period of 1 hour to 2 hours.

In a yet another embodiment, contact or exposure of the solution obtained upon passing the sized crude capsular polysaccharide solution through reinforced cellulose membrane having quaternary ammonium ligand, to SiO₂ is carried out at temperature of 50±5° C. for a period of 60 to 80 minutes.

In a preferred embodiment of the present invention, contact with SiO₂ is preferably carried out at a temperature ranging from 15° C. to 25° C. for a time period ranging from 60 minutes to 80 minutes under stirring.

In another embodiment of the present invention. SiO₂ was removed using centrifugation at 12000-14000g force.

In another embodiment of the present invention, common brand names of SiO₂ (silicon dioxide) available in the market are Acrosil®, Aeroperl® is used.

In another embodiment, the present invention involves treatment of purified capsular polysaccharide solution with activated carbon for removing color impurities. This treatment is carried out after exposure to SiO₂.

In an embodiment, the present invention provides a method for purification of capsular polysaccharides from Streptococcus pneumoniae of serotypes 1 and 5, wherein the said method comprises the steps of:
a. culturing the bacterial cells in standard growth conditions to obtain a fermentation broth;
b. lysing bacterial cells in fermentation broth obtained in step (a) to obtain a bacterial cell lysate comprising capsular polysaccharide, protein, nucleic acids, cell wall components and other impurities;
c. subjecting the bacterial cell lysate from step (b) to sizing using homogenizer to obtain a sized crude capsular polysaccharide solution;
d. passing the sized crude capsular polysaccharide solution obtained in step (c) through a reinforced cellulose membrane having quaternary ammonium as ligand; and
e. contacting the solution obtained after passing through reinforced cellulose membrane having quaternary ammonium as ligand, in step (d), with SiO₂ and isolating the capsular polysaccharide in a pure form.

In an embodiment, the present invention provides a method for purification of capsular polysaccharides, wherein the said method comprises the steps of:
a. culturing the bacterial cells in standard growth conditions to obtain a fermentation broth;
b. lysing bacterial cells in fermentation broth obtained in step (a) to obtain a bacterial cell lysate comprising capsular polysaccharide, protein, nucleic acids, cell wall components and other impurities;
c. subjecting the lysed broth to a preliminary purification process comprising of centrifugation, depth filtration, and diafiltration, to obtain a preliminary treated crude polysaccharide solution;
d. subjecting the preliminary treated crude polysaccharide solution from step (c) to sizing using homogenizer to obtain a sized crude capsular polysaccharide solution;
e. passing the sized crude capsular polysaccharide solution obtained in step (d) through a reinforced cellulose membrane having quaternary ammonium as ligand; and subsequently
f. contacting the solution obtained after passing through reinforced cellulose membrane having quaternary ammonium as ligand, in step (e), with SiO₂ and isolating the capsular polysaccharide in a pure form.

In an embodiment, the present invention provides a method for purification of capsular polysaccharides from Streptococcus pneumoniae of serotypes 1 and 5, wherein the said method comprises the steps of:
a. culturing the bacterial cells in standard growth conditions to obtain a fermentation broth;
b. lysing bacterial cells in fermentation broth obtained in step (a) to obtain a bacterial cell lysate comprising capsular polysaccharide, protein, nucleic acids, cell wall components and other impurities;
c. Subjecting the lysed broth to a preliminary purification process comprising of centrifugation, depth filtration, and diafiltration, to
d. subjecting the preliminary treated crude polysaccharide solution from step (c) to sizing using homogenizer to obtain a sized crude capsular polysaccharide solution;
e. passing the sized crude capsular polysaccharide solution obtained in step (d) through a reinforced cellulose membrane having quaternary ammonium as ligand; and subsequently
f. contacting the solution obtained after passing through reinforced cellulose membrane having quaternary ammonium as ligand, in step (e), with SiO₂ and isolating the capsular polysaccharide in a pure form.

In an embodiment, the diafiltration in step (c) above is carried out against phosphate buffer, using a 100 KDa MWCO cassette.

In an embodiment, the purified capsular polysaccharide solution obtained after contacting with SiO₂ can be further clarified using depth filter, carbon filter and/or 0.45-micron filter to obtain a filtered polysaccharide solution.

In an embodiment, the purified capsular polysaccharide solution can be further passed through reinforced cellulose membrane having quaternary ammonium as ligand to remove process impurities and obtain ultra-purified capsular polysaccharide solution.

In an embodiment, the present invention provides a method for purification of capsular polysaccharides, wherein the said method comprises the steps of:
a. culturing the bacterial cells in standard growth conditions to obtain a fermentation broth;
b. lysing bacterial cells in fermentation broth obtained in step (a) to obtain a bacterial cell lysate comprising capsular polysaccharide, protein, nucleic acids, cell wall components and other impurities;
c. Subjecting the lysed broth to a preliminary purification process comprising of centrifugation, depth filtration, and diafiltration, to
d. subjecting the preliminary treated crude polysaccharide solution from step (c) to sizing using homogenizer to obtain a sized crude capsular polysaccharide solution;
e. passing the sized crude capsular polysaccharide solution obtained in step (d) through a reinforced cellulose membrane having quaternary ammonium as ligand; and subsequently
f. contacting the solution obtained after passing through reinforced cellulose membrane having quaternary ammonium as ligand, in step
(c), with SiO₂ to obtain purified capsular polysaccharide solution; and
g. further passing the purified capsular polysaccharide solution, obtained after step (f) through reinforced cellulose membrane having quaternary ammonium as ligand, to remove process impurities and obtain ultra-purified capsular polysaccharide solution.

In an embodiment, the present invention provides a method for purification of capsular polysaccharides from Streptococcus pneumoniae of serotypes 1 and 5, wherein the said method comprises the steps of:
a. culturing the bacterial cells in standard growth conditions to obtain a fermentation broth;
b. lysing bacterial cells in fermentation broth obtained in step (a) to obtain a bacterial cell lysate comprising capsular polysaccharide, protein, nucleic acids, cell wall components and other impurities;
c. Subjecting the lysed broth to a preliminary purification process comprising of centrifugation, depth filtration, and diafiltration, to obtain a preliminary treated crude polysaccharide solution;
d. subjecting the preliminary treated crude polysaccharide solution from step (c) to sizing using homogenizer to obtain a sized crude capsular polysaccharide solution;
e. passing the sized crude capsular polysaccharide solution obtained in step (d) through a reinforced cellulose membrane having quaternary ammonium as ligand; and subsequently
f. contacting the solution obtained after passing through reinforced cellulose membrane having quaternary ammonium as ligand, in step
(e), with SiO₂ to obtain purified capsular polysaccharide solution; and
g. further passing the purified capsular polysaccharide solution, obtained after step (f) through reinforced cellulose membrane having quaternary ammonium as ligand, to remove process impurities and obtain ultra-purified capsular polysaccharide solution.

In a yet another embodiment, the purified capsular polysaccharide solution obtained after contacting with SiO₂ or the filtered polysaccharide solution obtained after depth filter, carbon filter and 0.45-micron filter treatment of the SiO₂ contacted purified polysaccharide solution, can be further subjected to purification by passing through reinforced cellulose membrane with quaternary ammonium ligand to remove process impurities.

In an embodiment, the purified capsular polysaccharide solution can be subjected to concentration and diafiltration using normal saline and water for injection to remove the low molecular weight polysaccharides and residual agents used in the purification process.

In a preferred embodiment, purified capsular polysaccharide solution can be subjected to concentration and diafiltration using 100 kDa molecular weight cut-off membrane.

The purified and ultra-filtered capsular polysaccharides can be further subjected to membrane filtration to remove bioburden. Usually, the capsular polysaccharide solution is filtered through a 0.45-micron filter followed by a 0.22-micron filter to remove the bioburden. However, any suitable membrane can be used for filtration to remove the bioburden. The filtered capsular polysaccharide solution can be refrigerated after sampling. Preferably, the capsular polysaccharide solution can be stored at −20° C. after sampling.

In another embodiment of the present invention, capsular polysaccharides in purified form have Protein Content of less than 2% of dry weight polysaccharide, Nucleic acid Content is less than 2% of dry weight polysaccharide; and endotoxin content is less than 0.5 IU/μg of dry weight polysaccharide.

In a preferred embodiment of the present invention, the capsular polysaccharides in purified form have Protein Content of less than 1% of dry weight polysaccharide, Nucleic acid Content is less than 1% of dry weight polysaccharide; and endotoxin content is less than 0.5 IU/μg of dry weight polysaccharide.

The product is characterized using sup. 1H-NMR data shows that the consistent with the chemical structure by the assignment of signals assigned to the protons of the polysaccharide molecule. The. 1H-NMR spectrum showed a series of well-resolved signals (protons from the methyl group) for the quantification of functional groups in the polysaccharide. Multi angle laser light scattering technique (MALLS) was used to profile the molecular size distribution of the polysaccharide. Purified polysaccharides comply with the WHO specifications.

In an embodiment, the capsular polysaccharides of the present invention can optionally be further subjected to sizing. The process of the present invention shows an additional advantage, wherein it helps in preventing the formation of high molecular weight polysaccharide aggregates by minimizing the intermolecular and intramolecular binding, thus allowing the sizing of the polysaccharides at much lower overall pressure.

In an embodiment, the purified capsular polysaccharide of the invention is used as an immunogen, with or without further modification, for use in immunization. For immunization purposes it is preferred to conjugate the capsular polysaccharide to a carrier molecule, such as a protein.

In an embodiment, the carrier proteins are selected from the group comprising of tetanus toxoid (TT), diphtheria toxoid (DT), CRM197, H. influenzae protein D, PhtX, PhtD, PhtDE fusions, detoxified pneumolysin, PorB, N19 protein, PspA, OMPC, toxin A or B of C. difficile and PsaA.

In a preferred embodiment, the present invention provides an immunogenic composition comprising purified capsular polysaccharide prepared according to the present invention individually conjugated to carrier protein selected from CRM197, PsaA or PspA.

In yet another preferred embodiment, the present invention provides an immunogenic composition comprising purified capsular polysaccharides from one or more serotypes 1, 2, 3, 4, 5, 6A, 6B, 7F, 8, 9N, 9V, 10A, 11A, 12F, 14, 15B, 17F, 18C, 19F, 19A, 20, 22F, 23F, and 33F conjugated to CRM197 carrier protein.

In an embodiment the immunogenic composition comprises a conjugated N. meningitidis serogroup Y capsular saccharide (MenY), and/or a conjugated N. meningitidis serogroup C capsular saccharide (MenC).

In another important embodiment of the present invention an immunogenic composition comprising purified capsular polysaccharide in pure form, conjugated to a carrier protein selected from diphtheria toxoid, tetanus toxoid, CRM 197, PsaA, and PspA.

In some embodiments, the present disclosure provides pneumococcal polysaccharide-protein conjugate vaccine compositions comprising pneumococcal polysaccharides having molecular weights ranging between about 100 kDa to about 400 kDa, about 125 kDa to about 425 kDa, about 150 kDa to about 450 kDa, about 175 kDa to about 475 kDa, about 200 kDa to about 500 kDa, about 250 kDa to about 550 kDa, about 300 kDa to about 600 kDa, about 100 kDa to about 1000 kDa, about 200 kDa to about 800 kDa, about 250 kDa to about 600 kDa, about 300 kDa to about 400 kDa, about 70 kDa to about 150 kDa, or about 75 kDa to about 125 kDa.

The following examples particularly describe the manner in which the invention is to be performed. But the embodiments disclosed herein do not limit the scope of the invention in any manner.

### EXAMPLES

Without limiting the scope of the present invention as described above in any way, the present invention has been further explained through the examples provided below.

The microorganisms used for study in the present invention have been obtained from American Type Culture Collection (ATCC), 12301 Parklawn Dr., Rockville, Md., U.S.A. 20852, and ATCC lists all of the serotypes of this invention as being freely available.

### Example 1: Purifying Crude Capsular Polysaccharide Only with SiO₂

The process involves diafiltration of crude polysaccharide obtained from lysing the bacterial cells using 100 kDa Molecular-weight cut-off (MWCO) membrane at the flow rate of 2-5 L/min. The polysaccharide is subjected to sizing using homogenizer. The polysaccharide from Serotype 1 is further subjected to CTAB treatment, wherein the CTAB is added to polysaccharide solution at 0.2% to 5%. After CTAB treatment, the pellet was separated by centrifugation and subsequently contacted with Aeroperl. For other serotypes, the sized polysaccharide solution, comprising of impurities, is subjected to Aeroperl solution (5.0-7.0 g/L) at incubation temperature of 20±5° C., for not less than 90 minutes. Further, Aeroperl is removed by centrifugation at 12,000-14,000g and polysaccharide shall be collected in the supernatant. The particle size of Aeroperl is in the range of 20-60 μm.

TABLE 1
Nucleic acid impurity levels before and after purifying
the capsular polysaccharides with Aeroperl.

| | |
|---|---|
| Serotype 1 | Serotype 5 |

| Before | After | Before | After |
|---|---|---|---|
| treatment | treatment | treatment | treatment |
| 23.91% | 2.39% | 20.61% | 6.51% |

It can be inferred from the data in Table 1 that Aeroperl treatment alone is ineffective in removing the impurities below the allowed regulatory levels.

### Example 2: Purifying the Crude Capsular Polysaccharide with Reinforced Cellulose Membrane with Quaternary Ammonium Ligand

The process involves diafiltration of crude capsular polysaccharide with impurities using 100 kDa Molecular-weight cut-off (MWCO) membrane at the flow rate of 2-5 L/min. The polysaccharide is subjected to sizing using homogenizer. Sized polysaccharide is passed through reinforced cellulose membrane with quaternary ammonium ligand and polysaccharide is collected in column flow through.

TABLE 2
Nucleic acid impurity levels before and after purifying
the capsular polysaccharides with reinforced cellulose
membrane with quaternary ammonium ligand

| | |
|---|---|
| Serotype 1 | Serotype 5 |

| Before | After | Before | After |
|---|---|---|---|
| treatment | treatment | treatment | treatment |
| 15.32% | 1.48% | 36.63% | 31.57% |

It can be inferred from the data in Table 2 that treatment with reinforced cellulose membrane, with quaternary ammonium ligand, alone is ineffective in removing the impurities below the allowed regulatory levels.

### Example 3: Purifying the Crude Capsular Polysaccharide with Reinforced Cellulose Membrane with Quaternary Ammonium Ligand and Further Exposing/Contacting to SiO₂

Streptococcus pneumoniae from serotype 1 and 5 fermentation broth is inactivated by adding sodium deoxy cholate 0.1-0.5% (DOC). Inactivated fermentation broth is subjected to centrifugation at 8000-10000g force with the flow rate of 200-600 LPH, and the supernatant is subjected to depth filtration, concentrated and diafiltered against phosphate buffer using 100 kDa MWCO cassettes for removal the cell debris. The pH of the clarified supernatant is adjusted to 5-6.

Concentrated crude capsular polysaccharide is filtered through a 0.45-micron filter and subjected to sizing using the high-pressure homogenizer. The concentrated sized crude polysaccharide solution is homogenized at a pressure range of 500-1000 bar, till the size of the polysaccharide reaches 400-700 kDa. Further, the pH of the crude sized capsular polysaccharide solution is adjusted in the range of 6.5-6.8 and the conductivity of the crude sized capsular polysaccharide solution is adjusted to 3±0.2 mS/cm.

Reinforced cellulose membrane having quaternary ammonium as ligand is initially cleaned with 0.5-1N NaOH, and subsequently equilibrated with phosphate buffered saline (PBS) till pH reaches 6.5-7.8 and conductivity reaches 3.2±0.3 mS/cm. PBS comprising of 10 mM phosphate in 15 mM NaCl solution is used for equilibrating the membrane. Sized crude capsular polysaccharide is passed through reinforced cellulose membrane having quaternary ammonium as ligand, at a flow rate of 2-5 L/min, and the flow-through solution, obtained upon passing the sized crude capsular polysaccharide solution through reinforced cellulose membrane having quaternary ammonium ligand, is then subjected to adsorption with Aeroperl for 1-2 hours at room temperature. The particle size of Aeroperl is in the range of 20-60 μm, and concentration in the range of 5-7%.

Aeroperl is subsequently separated from polysaccharide solution by centrifugation at 12000-14000g and filtration process using depth, carbon and 0.45-micron filters. Optionally, the treated solution obtained after Aeroperl treatment is further passed through reinforced cellulose membrane having quaternary ammonium ligand.

Process impurities are further removed from the filtered polysaccharide solution by again treating with reinforced cellulose membrane having quaternary ammonium as ligand. All the process impurities are selectively bound to the membrane. Polysaccharide solution is collected as flow through.

Purified polysaccharide is subjected to concentration and diafiltration using a 100 kD molecular weight cut off membrane using normal saline and water for injection to remove the low molecular weight polysaccharides and residual agents used in the purification process. Concentrated polysaccharide solution is filtered through 0.45 micron followed by 0.22 filter to remove the bioburden. Filter polysaccharide solution is stored at −20° C.

The product is characterized using sup. 1H-NMR data shows that the consistent with the chemical structure by the assignment of signals assigned to the protons of the polysaccharide molecule. The 1H-NMR spectrum showed a series of well-resolved signals (protons from the methyl group) for the quantification of functional groups in the polysaccharide. Multi angle laser light scattering technique (MALLS) was used to profile the molecular size distribution of the polysaccharide. Purified capsular polysaccharides comply with the WHO specifications.

TABLE 3
Impurity levels before and after treating the capsular
polysaccharide from S. pneumoniae Serotype 1, in
accordance with the process of the present invention:

| | | Serotype 1 | |
|---|---|---|---|
| | | Batch 1 | Batch 2 |
| Initial | Protein content | 5.47% | 7.02% |
| impurity | Nucleic Acid impurity | 29.78% | 15.32% |
| levels | Endotoxin content | <0.031 IU/μg | <0.031 IU/μg |
| Final | Protein impurity | <1% | <1% |
| impurity | | (<0.05 mg/mL) | (<0.05 mg/mL) |
| levels | Nucleic acid impurity | 0.15% | 0.11% |
| | Endotoxin content | Less than 0.031 | Less than 0.031 |
| | | IU/μg | IU/μg |

TABLE 4
Impurity levels before and after treating the capsular
polysaccharide from S. pneumoniae Serotype 5, in
accordance with the process of the present invention:

| | | Serotype 5 | |
|---|---|---|---|
| | | Batch 1 | Batch 2 |
| Initial | Protein content | 7.01% | 8.17% |
| impurity | Nucleic Acid impurity | 34.95% | 36.63% |
| levels | Endotoxin content | <0.031 IU/μg | <0.031 IU/μg |
| Final | Protein impurity | <1% | <1% |
| impurity | | (<0.05 mg/mL) | (<0.05 mg/mL) |
| levels | Nucleic acid impurity | 0.0887% | 0.2880% |
| | | | (0.0144 mg/ml) |
| | Endotoxin content | Less than | Less than |
| | | 0.031 IU/μg | 0.031 IU/μg |

It can be inferred from the data in Table 3 and 4 that treatment of capsular polysaccharide from S. pneumoniae Serotype 1 and 5, with reinforced cellulose membrane with quaternary ammonium ligand and further exposing to SiO₂, is effective in removing the impurities, much below the allowed regulatory levels, to obtain a superior product.

### ADVANTAGES

The present invention discloses a simple and cost-effective method to purify capsular polysaccharides, without the use of complex and expensive chromatography methods and toxic reagents like phenol.

Also, the process of the present invention is robust at commercial scale, requiring low-cost materials in the process and no specialized facilities or disposal of hazardous materials.

Further, the process of the present invention helps in preventing the formation of high molecular weight polysaccharide aggregates by minimizing the intermolecular and intramolecular binding. High molecular weight polysaccharide aggregates are difficult to size even at high pressure ˜2000 bar. Using the process of the present invention, capsular polysaccharides can be sized at much lower pressures. For instance, capsular polysaccharides can be sized at pressures of around 1200 bar or even lower.

We claim:

1. A method for purification of capsular polysaccharides, wherein the said method comprises steps of:
a. culturing the bacterial cells in standard growth conditions to obtain a fermentation broth:
b. lysing the bacterial cells in fermentation broth obtained in step (a) to obtain a bacterial cell lysate comprising capsular polysaccharide, protein, nucleic acids, cell wall components and other impurities:
c. subjecting the bacterial cell lysate from step (b) to sizing using homogenizer to obtain a sized crude capsular polysaccharide solution;
d. passing the sized crude capsular polysaccharide obtained in step (c), through a reinforced cellulose membrane having quaternary ammonium as ligand; and
e. contacting the solution obtained after passing through the reinforced cellulose membrane having quaternary ammonium, in step (d), with silicon dioxide (SiO₂) and isolating the capsular polysaccharide in a pure form.

2. The method claimed in claim 1, wherein the bacteria is selected from but not limited to Streptococcus, Staphylococcus, Enterococci, Bacillus, Corynebacterium, Listeria, Erysipelothrix, Clostridium, Haemophilus, Neisseria, or Klebsiella.

3. The method as claimed in claim 2, wherein the bacteria is selected from, but not limited to Streptococcus pneumoniae, Streptococcus pyogenes, Streptococcus agalactiae, Group C & G Streptococcii Staphylococcus aureus, Haemophilus influenzae, Neisseria meningitidis, or Klebsiella pneumoniae.

4. The method as claimed in claim 1, wherein the capsular polysaccharide is isolated from, but not limited to Streptococcus pneumoniae serotypes selected from the group comprising of serotypes 1, 2, 3, 4, 5, 6A, 6B, 6C, 6D, 6E, 6G, 6H, 7A, 7B, 7C, 7F, 8, 9A, 9L, 9F, 9N, 9V, 10A, 10B, 10C, 10D, 10F, 11A, 11F, 11B, 11C, 11D, 11E, 12A, 12B, 12F, 13, 14, 15A, 15C, 15B, 15F, 16A, 16F, 17A, 17F, 18C, 18F, 18A, 18B, 19A, 19B, 19C, 19F, 20, 20A, 20B, 21, 22A, 22F, 23A, 23B, 23F, 24A, 24B, 24F, 25F, 25A, 27, 28F, 28A, 29, 31, 32A, 32F, 33A, 33C, 33D, 33E, 33F, 33B, 34, 45, 38, 35A, 35B, 35C, 35F, 36, 37, 38, 39, 40, 41F, 41A, 42, 43, 44, 45, 46, 47F, 47A, and 48.

5. The method as claimed in claim 6, wherein the capsular polysaccharide is isolated from Streptococcus pneumoniae serotypes selected from serotypes 1 and 5.

6. The method as claimed in claim 1, wherein the purified capsular polysaccharide solution from step (e) is further treated with activated charcoal to remove colour impurities.

7. The method as claimed in claim 1, wherein the purified capsular polysaccharide from step (e) is further passed through reinforced cellulose membrane having quaternary ammonium as ligand, to remove process impurities.

8. The method as claimed in claim 1, wherein the purification method is an alcohol-free process and does not employ CTAB or other harsh chemicals.

9. The method as claimed in claim 1, wherein the reinforced cellulose membrane having quaternary ammonium ligand is equilibrated with buffer till pH reaches 6.5-7.5 and conductivity reaches 2-4 mS/cm.

10. The method as claimed in claim 1, wherein the pH of sized crude capsular polysaccharide solution is adjusted to a range of 6-10 and the conductivity of the sized crude capsular polysaccharide solution is adjusted to a range of 1.0 mS/cm to 5.0 mS/cm before passing the solution through reinforced cellulose membrane having quaternary ammonium ligand.

11. The method as claimed in claim 1, wherein the sized crude capsular polysaccharide solution from step (c) is passed through the reinforced cellulose membrane at a flow rate of 2-10 L/min, preferably in the range of 2-5 L/min.

12. The method as claimed in claim 1, wherein the particle size of SiO₂ used ranges from 0.01 μm to 200 μm.

13. The method as claimed in claim 12, wherein the particle size of SiO₂ is preferably in the range of 20 μm to 60 μm.

14. The method as claimed in claim 1, wherein the amount of SiO₂ used ranges from 0.5% to 20% (w/v).

15. The method as claimed in claim 14, wherein the amount of SiO₂ preferably ranges from 5% to 7% (w/v).

16. The method as claimed in claim 1, wherein the contacting with SiO₂, in step (e) is carried out at a temperature ranging from 15° C. to 60° C. for a period ranging from 10 minutes to 16 hours.

17. The method as claimed in claim 1, wherein SiO₂ was removed using centrifugation at 12000-14000g force.

18. The method as claimed in claim 1, wherein the fermented broth is lysed using a lytic agent such as anionic or cationic detergent.

19. The method as claimed in claim 1, wherein the lytic agent is selected from, but not limited to sodium deoxycholate (DOC), N-lauryl sarcosine (NLS), chenodeoxycholic acid sodium, and saponins.

20. An immunogenic composition comprising purified capsular polysaccharide, prepared according claim 1, conjugated to a carrier protein selected from diphtheria toxoid, tetanus toxoid, CRM 197, PsaA, and PspA.
